(19) United States
(12) Patent Application Publication
(10) Pub. No.: US 2024/0000405 A1
(43) Pub. Date: Jan. 4, 2024

(54) DENTAL ASSESSMENT USING SINGLE NEAR INFARED IMAGES

(71) Applicant: ILLUMENAR INC., Syracuse, NY (US)

(72) Inventors: Justin Monaco, Syracuse, NY (US); Jeffrey Monaco, Syracuse, NY (US); Anh Dao, Syracuse, NY (US); Bianca Louise Andrada, Syracuse, NY (US)

(21) Appl. No.: 18/212,481

(22) Filed: Jun. 21, 2023

Related U.S. Application Data
(60) Provisional application No. 63/353,905, filed on Jun. 21, 2022.

(51) Int. Cl. A61B 6/14; G06T 7/00

(52) U.S. Cl. CPC A61B 6/145; G06T 7/0012; G06T 2207/30036; G06T 2207/20081

(57) ABSTRACT

The present invention includes systems and methods for dental imaging using near infrared (NIR) light to illuminate a tooth (or teeth) using a sensor, such as a camera, to obtain a single image for each view of the tooth (or teeth) imaged for a dental assessment.

### FIELD OF THE INVENTION

This invention relates generally to the field of dental and oral imaging, including systems and methods for assessing one or more aspect of oral health.

### DRAWINGS

The patent or application file contains at least one drawing executed in color. Copies of this patent or patent application publication with color drawing(s) will be provided by the Office upon request and payment of the necessary fee.

### BACKGROUND

The 2019 Global Burden of Disease Study reported that oral diseases affect nearly 3.5 billion people worldwide. Oral diseases may cause pain, discomfort, disfigurement, malnutrition, and death. The Global Burden of Disease Study reported that dental caries (tooth decay) in permanent teeth was the most common health condition, not only among oral health conditions, but amongst all conditions studied. An estimated 2 billion adults and 520 million children suffer from caries of primary teeth.

Aside from practicing good oral hygiene, early detection of tooth decay is critical to limit and treat the damage caused by dental caries. A simple, visual-tactile inspection of teeth by a dentist or other professional is generally insufficient in detecting the early stages of dental caries. Studies estimate that dentists are only able to detect approximately 30% of early-stage dental caries lesions. Unfortunately, X-ray radiography, among the most commonly available dental imaging tools likewise is unable to detect early-stage dental caries, especially when the lesions are found within the enamel of a tooth.

Thus, by the time a carious lesion is detected by a visual inspection or X-ray have breached the enamel into the underlying dentin and/or otherwise require drilling and the application of a filling. Such outcomes are not only painful and expensive, but also discourage patients from seeking dental attention until the problem has manifested into a serious issue. In contrast, if a caries lesion is discovered early enough, it may be repaired using a remineralization procedure, which is far less invasive than a traditional filling.

In response to the limited detection provided by X-rays, and in order to limit the radiation exposure caused by imaging, several non-ionizing-radiation based dental imaging techniques have entered development. For example, magnetic resonance imaging (MM) has been used for dental imaging. However, its prohibitive cost and equipment requirements have limited its use. Optical coherence tomography (OCT) has also been studied for dental imaging, though its inability to scan deep within a tooth has limited its applicability.

Near-infrared (NIR) dental imaging has likewise been the focus of some development efforts. Like MRI and OCT imaging, NIR imaging has not reached wide-spread use. First, many reports in the field of dental imaging explain that NIR-based dental imaging techniques are inherently unreliable, as they are purportedly sensitive to stains on teeth. Thus, certain NIR-based methods require the use of a fluorescent dye (often administered by injection), which increases cost and limits its use to qualified professionals. Other NIR-based methods use NIR transillumination and thus require the use of bulky and complex equipment—often shaped like a mouthguard and including moveable cameras. Still other NIR-based methods require an imaging device to take a series of stacked images (generally at different NIR wavelengths), which are then combined and processed for every view obtained during imaging. As each tooth may require a number of different views for a proper assessment, the requirement for a stack of individually-obtained images for each view leads to long scan times. Moreover, combining and analyzing each stack of images necessitates large power and data-processing requirements.

The drawbacks of current dental imaging techniques have prevented their use as a widespread tool, useable by both professionals and consumers, for quick and simple oral health assessments.

### SUMMARY

The present invention includes systems and methods for dental imaging using near infrared (NIR) light to illuminate a tooth (or teeth) using a sensor, such as a camera, to obtain a single image for each view of the tooth (or teeth) imaged for a dental assessment. In preferred aspects, the invention includes systems that provide NIR illumination to a tooth about or along the same axis on which the sensor (e.g., a camera) obtains an image of the NIR-illuminated tooth. Thus, in contrast to prior transillumination NIR imaging systems, in which a tooth to be imaged is disposed between the illumination source and camera, systems of the invention may include both the NIR illumination source and camera on the same face of a device. This allows for systems of the invention to use compact illumination/imaging probes.

Further, in preferred aspects, systems and methods of the invention obtain only a single image for each view of a tooth/teeth required for a dental assessment. This departs from some prior approaches of NIR dental imaging, which require a stack of images to be obtained as a tooth is illuminated across a number of NIR wavelengths. Surprisingly, in contrast, the presently disclosed systems and methods are able to obtain clinically meaningful data of an NIR illuminated tooth from a single image. This reduces scan time and data processing requirements, and consequently provides systems of the invention with increased flexibility in their form and design.

In preferred aspects, systems of the invention use a small, handheld probe that includes an NIR illumination source (e.g., one or more LED) and a camera. Handheld probes of the invention may be manufactured using low-cost components and configured in roughly the size and shape of consumer-focused electronic toothbrushes. Accordingly, it is contemplated that systems of the invention may be used by individuals at home to perform a dental assessment. By periodically using systems of the invention, users may obtain longitudinal monitoring of their oral health. Systems of the invention may incorporate directed guidance to facilitate users in performing a dental assessment. The results of a dental assessment may be sent to one or more third parties, such as a dentist and/or insurance carrier. Systems of the invention may incorporate a human-in-the-loop to review, guide, or direct a user before, during, or after a dental assessment. For example, systems of the invention may alert a user or their dentist to an anomaly, such as a potential early-stage carious lesion, in order to direct a user to seek treatment.

In certain aspects, the present invention includes systems for assessing oral/dental health. An exemplary system of the invention includes an imaging probe with a proximal portion configured as a handle and a distal portion that includes an imaging head. The imaging head is dimensioned for insertion into the mouth of a user. The imaging system may include an imaging subsystem, which includes an illumination source. The illumination source provides NIR illumination to one or more teeth being imaged. The imaging head also includes an imaging sensor, e.g., a camera, that is operable to capture a single of image of the tooth/teeth illuminated by the NIR light from the illumination source. The system further includes an analysis subsystem in communication with the imaging subsystem. The analysis subsystem is operable to detect a carious lesion in the single image of an illuminated tooth.

In preferred systems of the invention, the illumination subsystem illuminates the at least one tooth with NIR light along an axis and the imaging subsystem detects NIR light about or along the same axis to produce the single image.

In certain aspects, the illumination source produces light across a spectrum that includes visible light and the NIR light, and includes a physical filter to capture the image with only the NIR light. The filter may be moveable between a first position, whereby the imaging subsystem is operable to capture the image of the tooth with the NIR light, and to a second position, whereby the imaging system images the tooth in the visible spectrum.

In certain aspects, systems of the invention may further include a non-NIR light source. The non-NIR light source may provide illumination in a visible spectrum, which may help a user guide the probe into position. In certain aspects, when the filter is moved into a first position, it blocks or otherwise prevents illumination by the non-NIR light source.

In certain systems of the invention, the imaging head is removable from the imaging probe. In such systems, the imaging probe may be configured to accept a plurality of different imaging heads. The imaging probe may also be configured such that the imaging head may be replaced with a different tool or attachment, such as a toothbrush, solution dispenser (e.g., rinse, mouthwash, wetting/drying agents, and detectable (e.g., fluorescent) dye).

In certain aspects, the analysis subsystem operates on a user device in wireless communication with the imaging probe.

In preferred aspects, the analysis subsystem includes a machine learning (ML) classifier trained to detect in NIR light images features correlated with oral health conditions, e.g., carious lesions. The analysis subsystem may also provide a user with guidance to position the imaging head into a proper orientation to obtain the single image. In certain aspects, the analysis subsystem is configured to identify a particular tooth of a user in the single image.

In preferred aspects, the analysis subsystem provides an output indicative of the probability of an oral health condition based on the single image. The analysis subsystem may provide an output to a third part, e.g., a clinician, at a remote site. The analysis subsystem may provide different outputs to different parties, e.g., a user, a medical professional, and an insurance carrier.

In certain aspects, the analysis subsystem is housed separate from the imaging probe, and the imaging probe and analysis subsystem are in wireless communication. In some systems of the invention, the analysis subsystem, or a portion thereof, may be housed on a user's mobile smart phone. In certain aspects, the analysis subsystem, or a portion thereof, is housed in a base station. The base station may be capable of wireless communication with a user's mobile smart phone.

In preferred systems of the invention, in the single image of the tooth, healthy enamel appears transparent. In preferred systems of the invention, in the single image of the tooth, lesions and/or defects in tooth structure appear dark. In preferred systems of the invention, in the single image of the tooth, healthy dentin appears opaque and/or white in color. In preferred systems of the invention, in the single image of the tooth, lesions, decay, and/or other abnormalities in tooth dentin appear dark. In preferred systems of the invention, in the single image of the tooth, impurities, fractures, and/or fillings appear as dark spots and/or very bright spots.

### BRIEF DESCRIPTION OF THE DRAWINGS

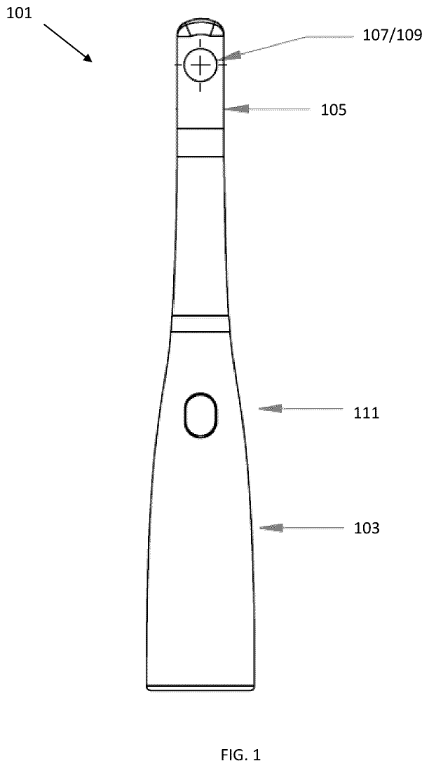

FIG. 1 shows a schematic of an imaging probe of the invention.

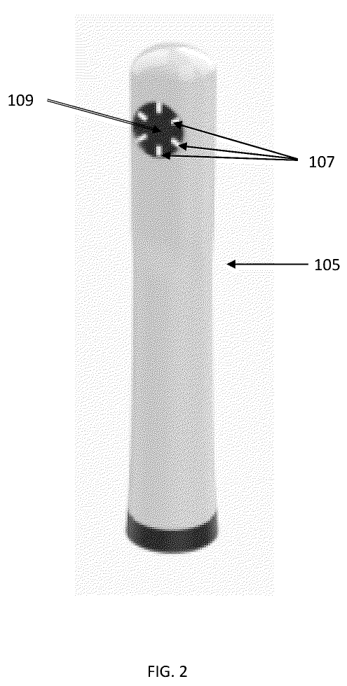

FIG. 2 shows a schematic of a removable imaging head to be used with an imaging probe of the invention.

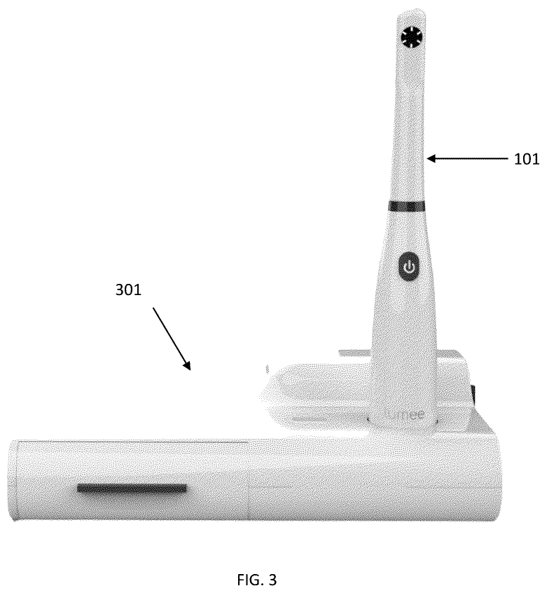

FIG. 3 shows an imaging probe with a base station.

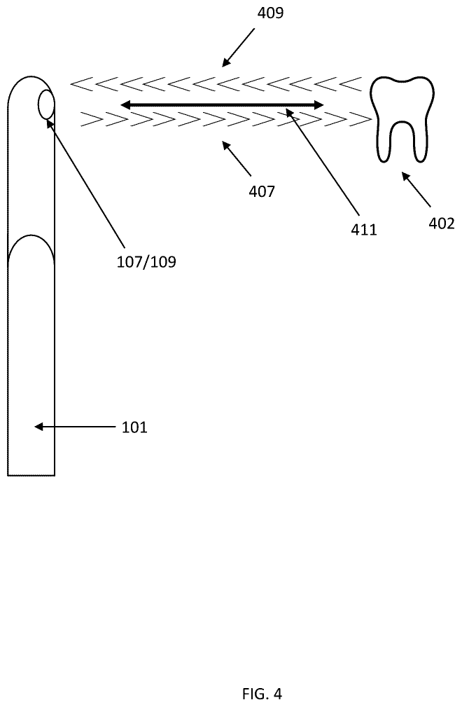

FIG. 4 shows a schematic of an imaging probe obtaining an NIR image of a tooth.

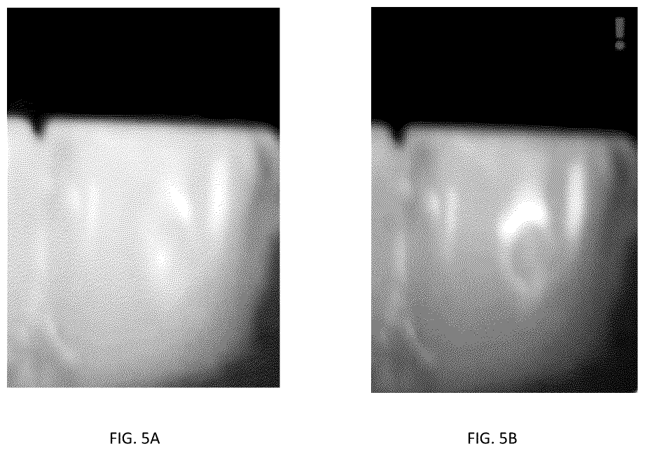

FIG. 5 shows the results of NIR imaging and assessment using an NIR imaging probe of the invention.

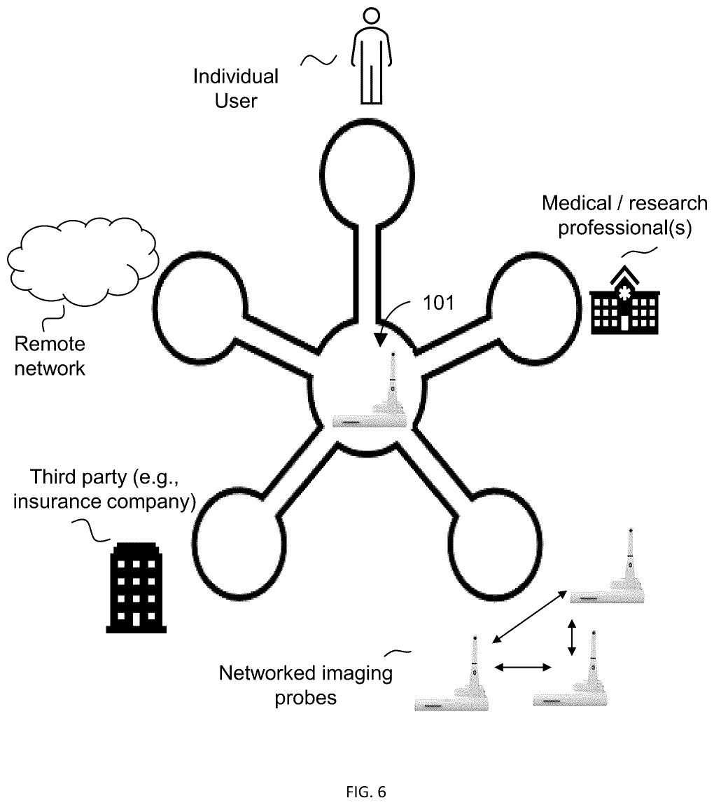

FIG. 6 shows potential machine learning network connections used in conjunction with the present invention.

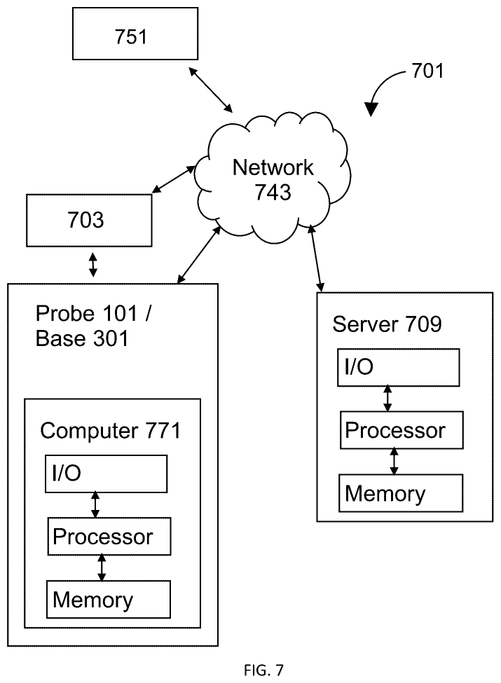

FIG. 7 shows a schematic of a system of the invention.

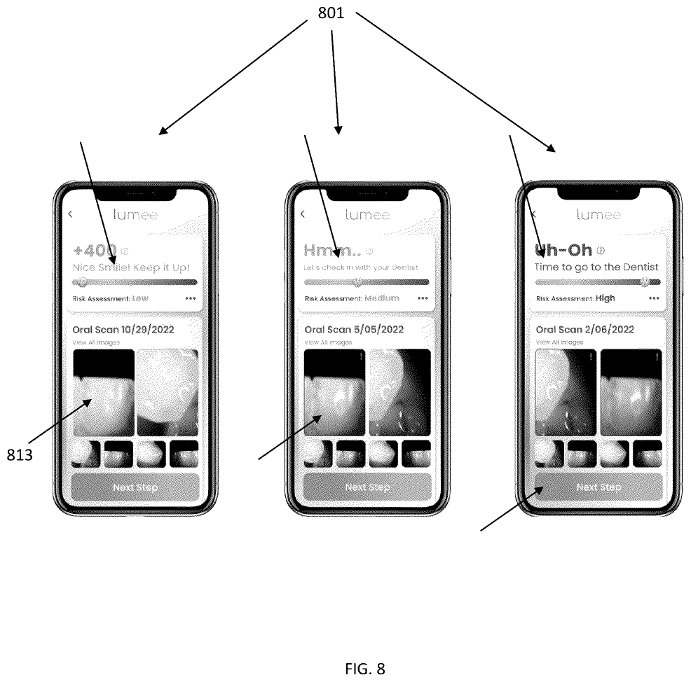

FIG. 8 shows a dental assessment report provided as an output by a system of the invention.

### DETAILED DESCRIPTION

The present invention includes systems and methods for dental imaging using near infrared (NIR) light to illuminate a tooth (or teeth) using a sensor, such as a camera, to obtain a single image for each view of the tooth (or teeth) imaged for a dental assessment. Unlike prior NIR light-based dental imaging techniques, the systems and methods of the invention are able to produce diagnostically meaningful information about dental health from a single image of a NIR illuminated tooth. Further, in preferred aspects, the invention includes systems that provide NIR illumination to a tooth about or along the same axis on which the sensor (e.g., a camera) obtains an image of the NIR-illuminated tooth. Thus, in contrast to prior transillumination NIR imaging systems, in which a tooth to be imaged is disposed between the illumination source and camera, systems of the invention may include both the MR illumination source and camera on the same face of a device. This allows for systems of the invention to use compact illumination/imaging probes.

FIG. 1 shows a schematic of an exemplary imaging probe 101 of the invention, which is used to obtain NIR images of teeth from within a user's mouth. As shown, the imaging probe is a small handheld device with a proximal portion configured as a handle 103, which may include one or more user controls 111. The exemplified device is similarly dimensioned to an electronic toothbrush. In this case, the handle is approximately 1 inch in diameter. The device also includes an imaging head 105, which is narrower than the handle for easy manipulation and adjustment within a user's oral cavity. Thus, the exemplified imaging probe includes a taper in its width as it extends distally towards the end of the imaging head, which has a diameter of approximately 0.3 inches in the illustrated probe.

The imaging probe 101 includes an imaging subsystem. In preferred aspects, components of the imaging subsystem are disposed in the imaging head 105. The imaging subsystem may include one or more NIR illumination source 107 (e.g., light emitting diodes (LED)) and one or more imaging sensor 109 (e.g., a camera). The illumination source 107 emits NIR illumination to a tooth, and the imaging sensor 109 produces a single image of the illuminated tooth as an output. The imaging probe may include or be in communication with an analysis subsystem that is operable to detect dental anomalies, e.g., an early-stage carious lesion, in the single image of an illuminated tooth.

FIG. 2 shows a close up view of an exemplary imaging head 105 of the imaging probe 101. As shown, in certain aspects, the illumination source 107 includes one or more sources of illumination. The illumination source 107 may include one or more of LEDs, photodiodes, a diode laser bar(s), a laser(s), a diode laser(s), fiber optics, a light pipe(s), halogen lights, and any other suitable light source. In preferred aspects, the illumination source includes one or more LEDs under the control of one or more printed circuit boards (PCB). In certain preferred aspects, the illumination source includes 2-8 LED lights that produce white, visible light and 2-8 LED lights that separately produce NIR illumination. In certain aspects, the illumination source may produce both NIR illumination and non-NIR illumination (e.g., visual light). Alternatively or additionally, systems of the invention may include devices with a plurality of illumination sources to produce spectrally distinct illumination.

In additional preferred aspects, the illumination source includes one or more of a laser(s), fiber optic(s), light pipe(s), and halogen bulb(s) to produce the NIR and/or non-NIR illumination. The present inventors discovered that, although LEDs produce high levels of controllable illumination, while using low power, certain LEDs individually cannot produce the requisite levels of NIR and/or non-NIR illumination for imaging. Consequently, in certain aspects, the illumination source includes a laser(s), fiber optic(s), light pipe(s), and/or halogen bulb(s), as these illumination devices produce an intense and controllable illumination using fewer sources relative to other modes of illumination. Thus, for example, a certain type of LED may require several individual LEDs in an array to produce the required NIR illumination for imaging as contemplated herein. In contrast, an illumination source that includes a single laser, fiber optic system, light pipe, or halogen bulb, may produce an equivalent level of illumination. Thus, while these devices may individually be larger than certain LEDs illumination sources, the ability to use less of them allows for the creation of devices with a smaller form.

As shown in FIG. 2, in certain systems of the invention, the imaging head is removable from the imaging probe. In such systems, the imaging probe may be configured to accept a plurality of different imaging heads. The imaging probe may also be configured such that the imaging head may be replaced with a different tool or attachment, such as a toothbrush, solution dispenser (e.g., rinse, mouthwash, wetting/drying agents, and detectable (e.g., fluorescent) dye).

FIG. 3 shows another view of the imaging probe 101, may be used in conjunction with a base station 301. The base station 301 may provide a convenient way to charge the imaging probe, such that it can be used wirelessly. Preferably, in order to make the imaging probe easier to manufacture as a water-resistant device, the imaging probe includes a battery that is charged using inductive charging. Preferably, the base station 301 includes a means to provide inductive power to an imaging probe when set or cradled on the base station. Alternatively or additionally, the imaging probe includes a batter that can be charged using a detachable cable. If both inductive and wired charging are provided, a user may, for example, charge the device when away from home and the base station.

In certain aspects, the base station 301 may include the analysis subsystem, which reduces the power requirements of the imaging probe itself. Thus, the imaging probe 101 may be in wireless communication with the base station 301. Alternatively or additionally, the imaging probe 101 may be in wireless communication with a user's smart telephone, e.g., via a software application and a Bluetooth™ connection.

FIG. 4 provides a schematic showing an imaging probe 101 of the invention imaging a tooth using NIR illumination. As shown, the illumination source(s) transmits NIR illumination 407 to a tooth 402. Preferably, the NIR illumination is transmitted at a wavelength in a range from 780-2500 nm. In certain aspects, the NIR illumination is transmitted at between 780 and 800 nm, between 800 and 820 nm, between 820 and 840 nm, between 840 and 860 nm, between 860 and 880 nm, between 880 nm and between 900 nm, between 900 nm and 925 nm, between 950 nm and 975 nm, between 975 nm and 1000 nm, between 1000 nm and 1100 nm, between 1100 nm and 1200 nm, between 1200 nm and 1300 nm, between 1400 nm and 1500 nm, between 1500 nm and 1600 nm, between 1600 nm and 1700 nm, between 1700 nm and 1800 nm, between 1800 nm and 1900 nm, between 1900 nm and 2000 nm, between 2000 nm and 2100 nm, between 2200 nm and 2300 nm, between 2300 nm and 2400 nm, or between 2400 nm and 2500 nm. In certain aspects, the NIR illumination is transmitted at one or specific NIR wavelengths. In certain aspects, the NIR illumination is transmitted across a range of NIR wavelengths.

During illumination of a tooth, the NIR light 407 transmitted to the tooth 402 is scattered, back scattered, reflected, and/or absorbed by various components of the tooth, e.g., dentin, enamel, and any anomalies therein. NIR light, after this scattering, back scattering, reflection, and/or absorption is detected by the imaging sensor 109.

As shown in FIG. 4, in preferred aspects, the imaging sensor 109 detects NIR light 409 transmitted from the illuminated tooth 402 along the same (or a similar) axis 411 as the path of travel of the NIR light 407 transmitted to the tooth 402 from the illumination source 107. In certain aspects, the NIR light is transmitted up to a 45-degree angle relative to the axis of light from the tooth to the imaging sensor. However, the most preferred embodiments include devices in which the axis of illumination and detection are, or about, coincident with one another.

Having a coincident path for the illuminating and detected light provides several advantages over NIR systems in which the tooth is disposed between a light source and sensor. First, the coincident axis prevents minimizes optical losses in the detected light. Further, although it causes problems when using large optics, small optics disposed near a sample surface are uniquely suited for producing and detecting coincident illumination and detected light. Thus, in the present application where large size and detection distances are undesirable, small optical components are desired in order to fit comfortably within an oral cavity. Further, the coincident axis permit both the imaging sensor(s) and illumination source(s) to be disposed on the same facet of an imaging head, such that they are both facing a tooth during imaging. This helps facilitate the compact sizes of the probes of the invention.

In preferred aspects, the imaging head 105 also includes one or more light filters. In even more preferred aspects, the imaging head 105 includes a physical light filter, e.g., a lens/lenses, a mirror/mirrors, slit(s), grid(s), and/or pinhole(s). Alternatively or additionally, the imaging head 105 includes a non-physical light filter (e.g., using software to parse out detected light that is not NIR light). The light filter may filter light being transmitted to the imaging sensor, such that only NIR light enters the imaging sensor. This becomes of some import when the device includes an illumination source that produces non-NIR light (e.g., light in the visual spectrum), which would interfere with producing an NIR light image. In certain aspects, a physical filter may be moveable between a first position by a user, whereby the imaging subsystem is operable to capture the image of the tooth with the NIR light, and to a second position, whereby the imaging system images the tooth in the visible spectrum.

In certain aspects, systems of the invention may further include a non-NIR light source. The non-NIR light source may provide illumination in a visible spectrum, which may help a user guide the probe into position. In certain aspects, when the filter is moved into a first position, it blocks or otherwise prevents illumination by the non-NIR light source.

In certain aspects, the light filter also filters light transmitted from the illumination source such that only light of one or more wavelengths are transmitted to a tooth. This may include transmitting light at a certain NIR wavelength, which is a different wavelength from detected NIR light allowed through the filter, thus reducing interference within the system.

As shown in FIG. 4, NIR light 409 reflected/scattered from the tooth is detected by the imaging sensor 109. Preferably, the imaging sensor includes a camera, such as a complementary metal-oxide-semiconductor (CMOS) camera or a scientific CMOS (sCMOS) camera. The imaging sensor may also include one or more objective, optical components, mirrors, filters and the like. The imaging sensor detects NIR light from an illuminated tooth to produce an NIR light image. The image is then sent to an analysis subsystem for a dental assessment. In certain aspects, the probe includes one or more sensors for NIR imaging. Alternatively or additionally, the probe includes one or more sensors for non-NIR imaging, such as a video camera to provide a live-feed of the probe's positioning such that it may be guided into a proper orientation.

In certain aspects, the imaging sensor includes or is coupled to one or more means (e.g., controllable optics) for adjusting the focus, zoom, and/or depth of field for the imaging sensor. In certain aspects, the imaging sensor includes a focusing adjustment capability to allow for imaging at different depths of focus. The control for the imaging capability may be under a user's control via a control on the body of the probe and/or an interface in a coupled software application. Unlike existing NIR-based dental imaging development efforts, systems and methods of the invention need obtain only a single image for each view of a tooth/teeth required for a dental assessment. This departs from some prior approaches of NIR dental imaging, which require a stack of images to be obtained as a tooth is illuminated across a number of NIR wavelengths. Surprisingly, in contrast, the presently disclosed systems and methods are able to obtain clinically meaningful data of an NIR illuminated tooth from a single image. This reduces scan time and data processing requirements, and consequently provides systems of the invention with increased flexibility in their form and design.

FIGS. 5A and 5B show single images of the same tooth obtained using an NIR device of the invention. The images were obtained several months apart, with the image in 5B being taken later. The NIR images provide detail due to different transmittances of teeth, skin tissue, and the like, for near-infrared radiation and clearly shows teeth and periodontal structures. Prior NIR development efforts dismissed the ability of NIR imaging to obtain clinically relevant information for a dental assessment from a single image. However, as shown in FIGS. 5A and 5B, the systems and methods of the invention provide meaningful data from single images.

During imaging, NIR light travels from the imaging head positioned above the tooth or teeth and is reflected back to the camera where healthy enamel appears transparent while lesions or defects in tooth structure appear dark (black or dark gray) due to the absorbance of light rather than reflectance of light. Healthy dentin appears opaque or white in color while non healthy (lesions, decay, any abnormality) dentin appears dark due to the absorbance of light rather than the reflectance of light. Impurities such as fractures or fillings falling out disperse light in which they show as dark spots or very bright spots due to the scattering of light back to the camera head or not in the camera field of view. As shown in FIGS. 5A-5B, during the short time between images, an early-stage carious lesion formed on the tooth, causing a detectable darkening in the imaged enamel and dentin, as clearly evident in the image.

In preferred aspects, systems of the invention include an analysis subsystem to analyze the single MR images to perform a dental assessment. Preferably, the analysis subsystem includes a machine learning (ML) classifier/model trained to detect in NIR light images features correlated with oral health conditions.

The systems and methods of the invention may employ ML classifiers trained using data sets that include annotated NIR dental images. These training images may be annotated to indicate the presence and/or absence of a dental anomaly, such as a carious lesion. The systems can be thus trained to correlate features in NIR dental images with dental anomalies and/or healthy teeth. These features may be imperceptible to a human technician analyzing an NIR, and yet may be associated with a particular disease or pathology by the classifier. Thus, in certain aspects, the NIR dental images are analyzed using one or more other dental assessment techniques, e.g., a visual inspection by a human technician. The results of the other-technique assessment(s) may be used to provide data for the training image annotations. For example, a human technician may discover a carious lesion in a tooth that is then imaged to produce a training NIR image. The NIR image may be annotated to highlight the presence of the carious lesion, such that the classifier correlates features in the NIR image to the annotated anomaly. Dental anomaly features in NIR images that correlated with those found in assessment of the same tooth using another method (e.g., visual inspection or X-ray radiograph) can serve to confirm and/or ground truth an assessment of the NIR image. Thus, the methods and systems of the invention can leverage data obtained from NIR imaging to improve the more commonly-available X-ray radiograph imaging.

In certain aspects, the present invention also includes ML systems trained using data from various sources separated by time and/or geography. The training data may include, for example, NIR dental images and known pathology results collated at a central source and remotely distributed to individual imaging probes via a networked connection.

Generally, ML systems have increased accuracy when trained using large data sets, and can continually improve with additional training data. In order to obtain this volume of data, it must come from distributed sources, such as various hospitals, research institutions, distributers of the imaging probes of the invention, and/or even from imaging probes used by consumers. However, in certain aspects, as the training data NIR images cultivated from individual users, to assure patient confidentiality and privacy, and in order to comply with relevant regulations such as the Health Insurance Portability and Accountability Act (HIPAA), confidential patient data should not leave their origin sources.

FIG. 6 shows an imaging probe with an operable ML classifier 101 connected to various locations that have the required training data. These locations (e.g., medical professionals, insurance companies, probe distributors, and other imaging probe users are separated from the ML classifier by time and/or geography. In certain aspects, distributed ML classifiers may be emplaced at these various locations and trained using local data.

The ML classifiers may be connected to, or receive data from, data stores at the various locations. These data stores may, for example, be picture archiving and communication systems (PACS). These subsystems can be computer hardware systems sent to the various locations, which include the ML classifier architecture. Advantageously, this provides a gap between the data archives at a location and the ML classifiers. Alternatively, the ML classifiers can be hosted on, or integrated into, computer systems at the various locations. As also shown in FIG. 6, in certain aspects imaging probes of the invention may form a dedicated network through which training images are passed to further train the ML classifiers connected to the network.

The trained ML classifiers can update a central ML system, for example, using a federated learning model. By using such an arrangement, the ML classifiers of the invention can be trained using data from distributed sources, while ensuring that confidential patient data does not leave a hospital or other research institution. Alternatively, or in addition, the ML classifiers may obtain data, such as NIR images, and scrub them of all private or confidential data, and send them to a ML classifier or another central image repository.

Moreover, in certain aspects, the ML classifiers are able standardize data from various locations to eliminate biases or artifacts attributable to different instruments, e.g., NIR imaging devices from different manufacturers, which may be used under diverse imaging conditions and/or parameters.

In certain aspects, the ML classifiers used in the invention are used to develop masks that can be applied to NIR images from different instruments, operating conditions and/or parameters. A different mask can be applied, for example, to data from different instruments. Applying the masks to the data from the different instruments standardizes the data obtained from those instruments.

In certain aspects, the ML classifier, analyzing a single NIR image of a tooth, provides a dental assessment as an output. A dental assessment, consistent with the definition provided by the American Dental Association, and as used herein may include an inspection of a tooth or teeth, using a single NIR image for each view of a tooth required, to identify possible signs of oral or systemic disease, malformation, or injury and the need for referral for diagnosis and/or treatment. In certain aspects, the dental assessment includes images and data providing an assessment of hard (cementum, dentin, enamel, dental caries) and/or soft (gums, roots, tongue, throat, etc.) dental tissue. In certain aspects, the dental assessment includes an assessment regarding any appliances of fillings in a user's mouth.

FIG. 8 shows the results of three dental assessments, taken at various times for the same teeth of the same patient, which as exemplified, is provided to a user's smart telephone via a wireless connection and a software application. As shown, the dental assessments display the single NIR image(s) 813 upon which the assessments were based. The display provides various types of information as at least one among text, an icon, a graphic, and an image. As shown, the assessments provided a graduated scale regarding the risk for a certain dental condition, in this case, a carious lesion. The report also contains an alert associated with a particular image of a tooth that includes a potential dental anomaly.

As the ML classifier's confidence increases regarding the detection of a dental anomaly, it may provide a user with varying follow up instructions. In certain aspects, the assessment or classifier includes an input controller (e.g., at least one button for generating commands such as a photographing command, an image generation command, a modification command, or the like). In FIG. 8, the dental assessment includes a “next step” command 811, which allows the user to, for example, contact their insurer, dentist, or schedule a consultation.

In certain aspects, the systems of the invention include a control module, which is preferably housed within a computer in the device. In certain aspects, the control module is a part of the analysis system and/or imaging system.

In certain aspects, the control module and/or imaging system provides a control signal for zooming in/zooming out, photographing, or the like of the imaging sensor (e.g., CMOS camera). The control signal for positioning, zooming in/zooming out, photographing, or the like of the optical sensor may be transmitted directly by the control module to the imaging system, and/or direct a user to take actions (e.g., positioning the probe within the mouth).

In certain aspects, the imaging probes 101, imaging probe base stations 301 and/or smart telephone of a user of the invention are capable of creating a wireless network link along which NIR images, data, and the like are transmitted. Additionally or alternatively, the software application may be configured to run on any computing device, including mobile devices. Such computing devices may include a smartphone, a tablet, a laptop, or any suitable computing device that may be used known in the art. The software application may be installed on a mobile device of a user. As a wireless communication protocol may be used, including any one among Bluetooth, Wi-Fi, Zigbee, ultra-wide band (UWB), and the like. NIR images, assessments, and instructions may be transmitted between components of the disclosed systems using this wireless connection.

In preferred aspects, the imaging probe 101 and/or imaging probe base station are capable of a wireless network connection to a user's smart telephone (or other mobile computing device) that hosts a software application that includes a graphical user interface (GUI) through which a user may interact with the imaging probe (e.g., via a control module and/or imaging system as described) and review NIR images and other data collected using the imaging probe.

In certain aspects, the GUI of the software application, preferably hosted on a user's mobile computing device (e.g., a smartphone) is configured provide the patient with instructions for using the imaging probe, including during NIR imaging. Instructions may include one or more of visual, textual, and/or audio guidance to aid the user in obtaining the requisite NIR images for a dental assessment.

In certain aspects, the GUI provides a user with an interface that provides a real-time video from the imaging probe. The video may be provided by a camera in the imaging probe head. This real-time video may be used, for example, to properly orient the imaging probe and/or perform a non-NIR assessment.

In certain cases, the application on the user's smartphone is in network communication with a software application at a third-party location, such as an insurance provider and/or a dental professional. In certain aspects, a third-party user may view live images/videos from a dental assessment and provide instructions to a user, for example, to reposition the imaging probe.

In certain aspects, users create user profiles on the software application. The user profile may include, for example, a user's dental and health information, dental provider, insurance information, demographic information, and the like. In certain aspects, the information in a user profile may be associated with NIR images obtained from an imaging probe of the invention. By creating profiles, users may link to their insurance provider and/or dentist to share NIR image data, e.g., dental caries identified in an NIR image by the image analysis system. In certain aspects, the user's profile includes a map or composite image of a user's teeth.

The map or composite image may be obtained with the single NIR images obtained during a health assessment(s). In certain aspects, the map or composite image is obtained using a non-NIR imaging device on the imaging probe (e.g., a non-NIR camera). In certain aspects, the map or composite image is provided by a user's dentist or using data provided from scans performed by a user's dentist (e.g., X-ray radiographs). The imaging probe of the invention may use such a map or composite image to calibrate the location of the probe to obtain a single image of a tooth from a same or similar view or perspective across multiple dental assessments. This also helps to ensure the appropriate user is being scanned with a particular profile.

In certain aspects, the imaging probe provides a real-time video or picture feed, preferably using a non-NIR camera on the probe, to help align the probe for an NIR image. In certain aspects, the real-time fee provides a bounding box, which indicates the focusing area for the NIR image. A users may select or change the focusing area by indicating the desired area in the live feed, e.g., using a couple smartphone with a touchscreen input. The display for the user may provide an indication, e.g., a change in the color of the bounding box, to indicate if the probe is in an appropriate orientation and/or focus for obtaining a particular NIR image.

In certain aspects, one or more image sensor of a probe as described herein, may provide a manual focus and/or autofocus mode for obtaining images. When the autofocus mode is chosen, a coupled imaging sensor on the probe, e.g., via instructions from a coupled control module, would automatically focus on the area of a tooth/teeth indicated by the bounding box. area. When the probe is at a proper orientation, which may be indicated by the probe to the user via an output as described herein, the control module may automatically focus the imaging sensor to obtain an NIR image. Optical control may be accomplished using optical components and/or software controls as known in the art. Once the focus within the bounding box is set, the control module may maintain focus on the area of the bounding box despite the user's minor movements of the probe. This ensures a stable image can be obtained using the probes. In certain aspects, once a tooth/teeth are in a proper focus, the control module may also assure that the tooth/teeth are at a proper magnification.

In certain aspects, the analysis subsystem of the imaging probe provides differing reports/outputs to different parties, which may be in a networked communication with the imaging probe, its base station, and/or the software application in networked communication with the imaging probe. For example, from the same single image(s), the systems of the invention may provide a consumer-facing report to an individual user, a technical report for their dentist, and a report with billing and authorization codes for an insurer.

For example, after a scan, a consumer-facing report may be provided to a user (e.g., via the software application GUI), such as that shown in FIG. 8, which provides a user with an indication that a potential anomalous dental condition was detected by the analysis subsystem. Data from that same scan may be sent to the user's dentist for further assessment. In certain aspects, the data is automatically forwarded, forwarded only after authorization by a user, and/or after authorization by a third-party payor. The data sent to the dentist may as a report that includes more technical information when compared to the consumer-facing report. For example, the report sent to a dentist may include prior images from longitudinal monitoring of a tooth/teeth, information from dental records, and the like.

Similarly, data from that same scan may be sent to a third-party payor, such as an insurance company. The insurance company may, for example, use data from that scan to record a user's compliance with a scanning program (e.g., as a part of a reimbursement program) or to pre-authorize consultation/treatment form the user's dentist. Alternatively or additionally, the third-party payor may use the data to recommend in-network dentists to a user for a follow-up appointment. The recommendation may, in part, be based on the data collected in the scan and qualifications of certain specialists to best address the condition. A report provided to a third-party payor may differ form that provided to a user or dental professional. For example, the report may be made using medical billing codes and/or Current Dental Terminology (CDT) codes, which is maintained by the American Dental Association and contains all dental procedure codes used by insurance companies in the United States.

In certain aspects, the systems of the invention may produce data used for demographics, population statistics, and/or public health purposes. In such cases, personal information will never leave a user's imaging probe. Data useful for public health may include, for example, data regarding the prevalence of anomalous dental conditions in a population. Data may also include that regarding dental hygiene practices when the device is compatible with a toothbrush head as described below. Further, in certain aspects, an imaging probe of the invention includes or is compatible with an oral thermometer. Thus, a user's body temperature may be recorded. Realtime information regarding the prevalence of fever in a population may provide a tool to help track the spread of certain diseases or outbreaks in a population.

In certain embodiments the imaging probe of the invention includes a removable imaging probe head. Thus, in such devices, the imaging probe may be replaced. In certain aspects, each different imaging head used with an imaging probe is associated with a different user. Different users may be associated with different profiles on a software application or different installations of the software application. The imaging head may include an electronic identifier, such that when attached to an imaging probe and powered, the imaging probe automatically provides NIR image data to an appropriate user profile or installation of the software application.

In certain aspects, the imaging probe head may be replaced with a different dental implement, such as a toothbrush or a different type of dental imaging or scanning device. When a user wishes to obtain a NIR image, they may replace a toothbrush head with the imaging head.

In certain aspects, the different dental implement is an electronic toothbrush. Thus, the body of the imaging probe may include, in addition to the elements of the imaging and/or analysis systems, elements configured to operate an electronic toothbrush, e.g., a motor, motor controller, etc. In certain aspects, the imaging probe recognizes what type of head is affixed to it (e.g., an NIR imaging head or an electronic toothbrush head). Upon recognition of the type of head, the imaging probe may provide a user with an appropriate control and functionality, such as providing the ability to take images or adjust the speed of an electronic toothbrush.

In certain aspects, like an imaging head, a dental implement (such as a toothbrush head, flossing head, etc.) may be linked to a user's profile or installation of the application. In this way, a user's dental hyenine and compliance may be recorded via the networked connection of the imaging probe to the software application. The software application may likewise provide a user with instructions and recommendations regarding use of the electronic toothbrush, e.g., a timer, an indication of whether certain teeth still need to be brushed, a brushing schedule, use of mouthwash or floss, or product recommendations.

In certain aspects, the additional or other dental implement includes a flossing head, such as a water-pressure-based flossing head or a mechanical flossing head. As with the toothbrush, a flossing head may be linked to the software application for directions and tracking.

Machine learning, as described herein, is branch of computer science in which machine-based approaches are used to make predictions. See Bera, 2019, “Artificial intelligence in digital pathology—new tools for diagnosis and precision oncology”, Nat Rev Clin Oncol 16(11):703-715, incorporated by reference. ML-based approaches involve a system learning from data fed into it, and use this data to make and/or refine predictions. As a generalization, a ML classifier/model learns from examples fed into it. Id. Over time, the ML model learns from these examples and creates new models and routines based on acquired information. Id. As a result, an ML model may create new correlations, relationships, routines or processes never contemplated by a human. A subset of ML is deep learning (DL). DL uses artificial neural networks. A DL network generally comprises layers of artificial neural networks. Id. These layers may include an input layer, an output layer, and multiple hidden layers. Id. DL has been shown to learn and form relationships that exceed the capabilities of humans.

By combining the ability of ML, including DL, to develop novel routines, correlations, relationships and processes amongst vast data sets including NIR dental images and patients' pathologies, clinical outcomes and diagnoses, the methods and systems of the disclosure can provide accurate diagnoses, prognoses, and treatment suggestions tailored to specific patients and patient groups afflicted with a various dental and oral health issues, including early-stage carious lesions.

Using the objective nature of ML, dental health assessments can be improved using the systems and methods of the disclosure. This includes using ML predictions as a companion to the decision making of trained specialists, or using ML to create independent predictions. Advantageously, ML models can be trained in such a way that they do not have preconceived notions of human specialists, and thus correlate certain image features without the inherent bias of a human.

ML systems of the invention can be trained with data sets that contain NIR dental images and known patient outcomes, to identify features within the images in an unsupervised manner and to create a map of outcome probabilities over the features. The ML models can receive images from patients, identify within the images predictive features learned from the training steps and locate the predictive features on the map of outcome probabilities to provide a prognosis or diagnosis.

This finds particular use in longitudinal monitoring of users' ongoing dental health. This process can be iterated over time to determine, for example, a subject's response to treatment.

ML systems of the disclosure can analyze NIR dental images and detect features based on, for example, pixel intensity and whether the pixel intensity meets a certain threshold. During ML training, these results can be confirmed and compared to those of human specialists viewing the same images.

FIG. 7 shows a computer system 701, preferably in communication with or as part of the imaging probe itself or its base 301, that may include an ML classifier 703 of the invention. The system 701 includes at least computer with a processor coupled to a memory subsystem including instructions executable by the processor to cause the system to analyze a single NIR dental image obtained using an imaging probe 101 to produce a dental health assessment as an output.

The system 701 includes at least one computer 771. Optionally, the system 701 may further include one or more of a server computer 709, which can include the ML classifier 703, and/or optionally one or more networked ML models 751 which may be distributed at various locations. Each computer in the system 701 includes a processor coupled to a tangible, non-transitory memory device and at least one input/output device. The system 701 includes at least one processor coupled to a memory subsystem.

The system 701 may include one or more PACS for storing and manipulating NIR dental images. The PACS may also store training data in accordance with the present disclosure. The PACS may be located at a hospital or other research institution, including a user's chosen dental professional.

The components (e.g., computer, server, PACS, and assay instruments) may be in communication over a network 743 that may be wired or wireless and wherein the components may be remotely located. Using those mechanical components, the system 701 is operable to receive or obtain training data such (e.g., annotated NIR dental images) for analysis. The system may use the memory to store the received data as well as the machine learning system data which may be trained and otherwise operated by the processor.

Processor refers to any device or system of devices that performs processing operations. A processor will generally include a chip, such as a single core or multi-core chip (e.g., 12 cores), to provide a central processing unit (CPU). In certain embodiments, a processor may be a graphics processing unit (GPU) such as an NVidia Tesla K80 graphics card from NVIDIA Corporation (Santa Clara, CA). A processor may be provided by a chip from Intel or AMD. A processor may be any suitable processor such as the microprocessor sold under the trademark XEON E5-2620 v3 by Intel (Santa Clara, CA) or the microprocessor sold under the trademark OPTERON 6200 by AMD (Sunnyvale, CA). Computer systems of the invention may include multiple processors including CPUs and or GPUs that may perform different steps of methods of the invention.

The memory subsystem may contain one or any combination of memory devices. A memory device is a mechanical device that stores data or instructions in a machine-readable format. Memory may include one or more sets of instructions (e.g., software) which, when executed by one or more of the processors of the disclosed computers can accomplish some or all of the methods or functions described herein. Preferably, each computer includes a non-transitory memory device such as a solid-state drive, flash drive, disk drive, hard drive, subscriber identity module (SIM) card, secure digital card (SD card), micro-SD card, or solid-state drive (SSD), optical and magnetic media, others, or a combination thereof.

Using the described components, the system 701 is operable to produce a report and provide the report to a user via an input/output device. The output may include the predictive output, such as a dental health assessment. An input/output device is a mechanism or system for transferring data into or out of a computer. Exemplary input/output devices include a video display unit (e.g., a liquid crystal display (LCD) or a cathode ray tube (CRT)), a printer, an alphanumeric input device (e.g., a keyboard), a cursor control device (e.g., a mouse), a disk drive unit, a speaker, a touchscreen, an accelerometer, a microphone, a cellular radio frequency antenna, and a network interface device, which can be, for example, a network interface card (NIC), Wi-Fi card, or cellular modem.

Any of several suitable types of machine learning, including those set forth below, may be used for one or more steps of the disclosed methods and used in the systems of the invention. Suitable machine learning types may include neural networks, decision tree learning such as random forests, support vector machines (SVMs), association rule learning, inductive logic programming, regression analysis, clustering, Bayesian networks, reinforcement learning, metric learning, and genetic algorithms. One or more of the machine learning approaches (aka type or model) may be used to complete any or all of the method steps described herein.

For example, one model, such as a neural network, may be used to complete the training steps of autonomously identifying features in NIR dental images and associating those features with certain pathologies. Once those features are learned, they may be applied to test samples by the same or different models or classifiers (e.g., a random forest, SVM, regression) for the correlating steps. In certain embodiments, features may be identified using one or more machine learning systems and the associations may then be refined using a different machine learning system. Accordingly, some of the training steps may be unsupervised using unlabeled data while subsequent training steps (e.g., association refinement) may use supervised training techniques such as regression analysis using the features autonomously identified by the first machine learning system.

In certain aspects, the ML model(s) used incorporate decision tree learning. In decision tree learning, a model is built that predicts that value of a target variable based on several input variables. Decision trees can generally be divided into two types. In classification trees, target variables take a finite set of values, or classes, whereas in regression trees, the target variable can take continuous values, such as real numbers. Examples of decision tree learning include classification trees, regression trees, boosted trees, bootstrap aggregated trees, random forests, and rotation forests. In decision trees, decisions are made sequentially at a series of nodes, which correspond to input variables. Random forests include multiple decision trees to improve the accuracy of predictions. See Breiman, 2001, “Random Forests”, Machine Learning 45:5-32, incorporated herein by reference. In random forests, bootstrap aggregating or bagging is used to average predictions by multiple trees that are given different sets of training data. In addition, a random subset of features is selected at each split in the learning process, which reduces spurious correlations that can results from the presence of individual features that are strong predictors for the response variable. Random forests can also be used to determine dissimilarity measurements between unlabeled data by constructing a random forest predictor that distinguishes the observed data from synthetic data. Also see Horvath, 2006, “Unsupervised Learning with Random Forest Predictors”, J Comp Graphical Statistics 15(1):118-138, incorporated by reference. Random forests can accordingly by used for unsupervised machine learning methods of the invention.

In certain aspects, the ML model(s) used incorporate SVMs. SVMs are useful for both classification and regression. When used for classification of new data into one of two categories, such as having a disease or not having the disease, an SVM creates a hyperplane in multidimensional space that separates data points into one category or the other. SVMs can also be used in support vector clustering to perform unsupervised machine learning suitable for some of the methods discussed herein. See Ben-Hur, A., et al., (2001), “Support Vector Clustering”, Journal of Machine Learning Research, 2:125-137, incorporated by reference.

In certain aspects, the ML model(s) used incorporate regression analysis. Regression analysis is a statistical process for estimating the relationships among variables such as features and outcomes. It includes techniques for modeling and analyzing relationships between multiple variables. Parameters of the regression model may be estimated using, for example, least squares methods, Bayesian methods, percentage regression, least absolute deviations, nonparametric regression, or distance metric learning.

Bayesian networks are probabilistic graphical models that represent a set of random variables and their conditional dependencies via directed acyclic graphs (DAGs). The DAGs have nodes that represent random variables that may be observable quantities, latent variables, unknown parameters or hypotheses. Edges represent conditional dependencies; nodes that are not connected represent variables that are conditionally independent of each other. Each node is associated with a probability function that takes, as input, a particular set of values for the node's parent variables, and gives (as output) the probability (or probability distribution, if applicable) of the variable represented by the node. See Charniak, 1991, “Bayesian Networks without Tears”, AI Magazine, p. 50, incorporated by reference.

The machine learning classifiers of the invention may include neural networks that are deep-learning neural networks, which include an input layer, an output layer, and a plurality of hidden layers.

A neural network, which is modeled on the human brain, allows for processing of information and machine learning. A neural network may include nodes that mimic the function of individual neurons, and the nodes are organized into layers. The neural network includes an input layer, an output layer, and one or more hidden layers that define connections from the input layer to the output layer. The nodes of the neural network serve as points of connectivity between adjacent layers. Nodes in adjacent layers form connections with each other, but nodes within the same layer do not form connections with each other.

The system may include any neural network that facilitates machine learning. The system may include a known neural network architecture, such as GoogLeNet (Szegedy, et al., “Going deeper with convolutions”, in CVPR 2015, 2015); AlexNet (Krizhevsky, et al., “Imagenet classification with deep convolutional neural networks”, in Pereira, et al. Eds., “Advances in Neural Information Processing Systems 25”, pages 1097-3105, Curran Associates, Inc., 2012); VGG16 (Simonyan & Zisserman, “Very deep convolutional networks for large-scale image recognition”, CoRR, abs/3409.1556, 2014); or FaceNet (Wang et al., Face Search at Scale: 90 Million Gallery, 2015), each of the aforementioned references are incorporated by reference.

The systems of the invention may include ML models using deep learning. Deep learning (also known as deep structured learning, hierarchical learning or deep machine learning) is a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower-level features to form a hierarchical representation. Those features are preferably represented within nodes as feature vectors.

Deep learning by the neural network may include learning multiple levels of representations that correspond to different levels of abstraction; the levels form a hierarchy of concepts. In most preferred embodiments, the neural network includes at least 5 and preferably more than 10 hidden layers. The many layers between the input and the output allow the system to operate via multiple processing layers. Using deep learning, an observation (e.g., an image) can be represented in many ways such as a vector of intensity values per pixel, or in a more abstract way as a set of edges, regions of particular shape, etc. Those features are represented at nodes in the network. Preferably, each feature is structured as a feature vector, a multidimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

Systems and methods of the disclosure may use convolutional neural networks (CNN). A CNN is a feedforward network comprising multiple layers to infer an output from an input. CNNs are used to aggregate local information to provide a global predication. CNNs use multiple convolutional sheets from which the network learns and extracts feature maps using filters between the input and output layers. The layers in a CNN connect at only specific locations with a previous layer. Not all neurons in a CNN connect. CNNs may comprise pooling layers that scale down or reduce the dimensionality of features. CNNs follow a hierarchy and deconstruct data into general, low-level cues, which are aggregated to form higher-order relationships to identify features of interest. CNNs predictive utility is in learning repetitive features that occur throughout a data set. The systems and methods of the disclosure may use fully convolutional networks (FCN). In contrast to CNNs, FCNs can learn representations locally within a data set, and therefore, can detect features that may occur sparsely within a data set. The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for assessing oral health, the system comprising:
an imaging probe comprising a proximal portion configured as a handle and a distal portion comprising an imaging head dimensioned for insertion into an oral cavity of a user;
an imaging subsystem carried within the imaging head, the imaging subsystem comprising an illumination source positioned to illuminate a tooth within the oral cavity with near infrared (NIR) light and an image sensor operable to capture a single image of the tooth illuminated with the NIR light; and
an analysis subsystem in communication with the imaging subsystem and operable to detect a dental lesion in the single image of the tooth.

2. The system of claim 1, wherein the illumination subsystem illuminates the at least one tooth with NIR light along an axis and the imaging subsystem detects NIR light along the same axis to produce the single image.

3. The system of claim 2, wherein the illumination source produces light across a spectrum that includes visible light and the NIR light, and includes a physical filter to capture the image with only the NIR light.

4. The system of claim 3, wherein the filter is moveable between a first position whereby the imaging subsystem is operable to capture the image of the tooth with the NIR light and a second position whereby the imaging system images the tooth in the visible spectrum.

5. The system of claim 4, wherein the system further comprises a non-NIR light source.

6. The system of claim 5, wherein the non-NIR light source provides illumination in a visible spectrum.

7. The system of claim 5, wherein when the filter is moved into a first position, it blocks or otherwise prevents illumination by the non-NIR light source.

8. The system of claim 1, wherein the imaging head is removable from the imaging probe.

9. The system of claim 8, wherein the imaging probe is configured to accept a plurality of different imaging heads.

10. The system of claim 8, wherein the imaging probe is configured such that the imaging head may be replaced with a different tool.

11. (canceled)

12. The system of claim 2, wherein the analysis subsystem operates on a user device in wireless communication with the imaging probe.

13. The system of claim 12, wherein the analysis subsystem comprises a machine learning (ML) classifier trained to detect in NIR light images features correlated with oral health conditions, and wherein the analysis subsystem provides a user with guidance to position the imaging head into a proper orientation to obtain the single image, wherein the analysis subsystem identifies a particular tooth of a user in the single image, wherein the analysis subsystem provides an output indicative of the probability of an oral health condition based on the single image, wherein the analysis subsystem provides an output to a clinician at a remote site, and/or wherein the analysis subsystem is housed separate from the imaging probe, and wherein the imaging probe and analysis subsystem are in wireless communication.

14.-18. (canceled)

19. The system of claim 13, wherein the analysis subsystem is housed on a user's mobile smart phone.

20. The system of claim 13, wherein the analysis subsystem is housed in a base station.

21. The system of claim 20, wherein the base station is capable of wireless communication with a user's mobile smart phone.

22. The system of claim 1, wherein in the single image of the tooth, healthy enamel appears transparent.

23. The system of claim 22, wherein in the single image of the tooth, lesions and/or defects in tooth structure appear dark.

24. The system of claim 1, wherein in the single image of the tooth, healthy dentin appears opaque and/or white in color.

25. The system of claim 1, wherein in the single image of the tooth, lesions, decay, and/or other abnormalities in tooth dentin appear dark.

26. The system of claim 25, wherein in the single image of the tooth, impurities, fractures, and/or fillings appear as dark spots and/or very bright spots.
